(19) United States
(12) Patent Application Publication
(10) Pub. No.: US 2020/0093858 A1
(43) Pub. Date: Mar. 26, 2020

(54) VAGINAL BIOADHESIVE BORIC ACID FORMULATION AND ITS PREPARATION METHOD

(71) Applicant: Halo Science LLC, Morganville, NJ (US)

(72) Inventors: Yu Hui, Winston-Salem, NC (US); Xudong Yuan, Morganville, NJ (US); Tian Zhang, Richmond (CA)

(21) Appl. No.: 16/586,815

(22) Filed: Sep. 27, 2019

Related U.S. Application Data
(63) Continuation-in-part of application No. 15/294,724, filed on Oct. 15, 2016, now abandoned.

(30) Foreign Application Priority Data
Oct. 24, 2015 (CN) 201510518140.2

(51) Int. Cl. A61K 33/22; A61K 47/32; A61K 47/36; A61K 47/10; A61K 9/00; A61P 31/10; A61P 31/04; A61K 9/06; A61K 9/02; A61K 9/48

(52) U.S. Cl. CPC A61K 33/22; A61K 47/32; A61K 47/36; A61K 47/10; A61K 9/0034; A61K 9/4858; A61P 31/04; A61K 9/06; A61K 9/02; A61K 9/4866; A61P 31/10

(57) ABSTRACT

This present disclosure discloses a bioadhesive boric acid formulation and its use and preparation method. Specifically, a preferred vaginal bioadhesive boric acid formulation mainly comprises of: methylparaben, propylparaben, potassium sorbate, chitosan, glycerin, carbopol 974, polycarbophil, boric acid, and purified water. The vaginal boric acid preparation are capable of retaining and releasing boric acid in the vagina. It is proved experimentally that boric acid acts as an antibacterial component of this gel and can be maintained at a relatively high concentration in vaginal fluid. Further, it is demonstrated that the vaginal bioadhesive boric acid formulation has a dynamic viscosity >100000 cps, pH value of 2-5, good moisture retention capability. The formulation is non-allergenic, non-vaginal irritation, and capable of inhibiting bacteria and fungi.

### CROSS-REFERENCES TO RELATED APPLICATIONS

This application is a continuation-in-part application of U.S. patent application Ser. No. 15/294,724, filed Oct. 15, 2016, which claims the benefit of Chinese Patent Application No. 201510518140.2, filed Oct. 24, 2015, which applications are incorporated herein by reference in their entireties.

### BACKGROUND

### Technical Field

The present disclosure relates to a vaginal bioadhesive boric acid formulation and its preparation method. Vaginal bioadhesive boric acid formulation is a new formulation of boric acid. It uses bioadhesive polymer material as skeleton structure, retaining and releasing boric acid in vaginal fluid as an antibacterial ingredient. The disclosure is related to pharmaceutics, analytical chemistry, and microbiology.

### Description of the Related Art

About 80-95% of vaginitis originates from the infection of Candida albicans. T. glabrata infections accounted for only 5%-15% of vaginitis, but the treatment options are very limited. There are only a few reports in the literature about the treatments of T. glabrata vaginitis.

However, the incidence of T. glabrata is constantly increasing. This is probably because of the overuses of systemic oral conazoles, short-term treatment regimens and non-prescription topical antifungal therapies.

Most of T. glabrata vaginitis are chronic and relapsing. These vaginal fungi are increasingly resistant to imidazole and conazoles. The lowered sensitivity of T. glabrata to these drugs may be the result of the selection effects of the antifungal agents for mutated T. glabrata.

It was reported that boric acid concentration at 10-20 mg/mL in urine could suppress almost all common fungi or bacteria.

In an in vitro study, 4% of boric acid inhibited the proliferation of 2×106 per mL of C. albicans. Although boric acid has not been widely studied as a treatment for fungal vaginitis, it was reported that boric acid treatment of vulvovaginal candidiasis resulted in a cure rate of >90%. Boric acid treatment for 14 days was equivalent to conazoles.

For these reasons, it was recommended that boric acid be used as first-line treatment of refractory T. glabrata vaginitis. Clinical efficacy of boric acid can reach as high as 81%, while mycological eradication rate can reach 77%.

The use of boric acid 600 mg per day for a therapeutic course of two weeks did not cause adverse reactions. But genital burning sensation in some patients was common. Recurrent fungal vaginitis, can be treated with boric acid capsules twice a week for 1-3 months. 600 mg capsule formulation is usually an extemporaneous preparation by pharmacy, because boric acid vaginal formulation has not been approved for marketing and the commercial products are not available for clinicians and patients.

Bacterial vaginosis is the most common vaginal infection. Although antibiotic therapy against anaerobic bacteria can effectively relieve short-term symptoms, recurrence rate of approximately 30% after 3 months of therapy is largely frustrating.

There are reports for antibiotic maintenance therapy, such as the use of 0.75% of vaginal metronidazole gel twice a week. Despite not completely curative, the therapy reduced the recurrence of bacterial vaginosis. Unfortunately, had patients stopped using this suppressive maintenance therapy, the bacterial vaginosis would relapse.

It was hypothesized that the formation of G. vaginalis biological biofilm on the vaginal epithelial wall antagonizes antibacterial effect of metronidazole. The topical application of boric acid for vaginal antimicrobial therapy can promote biofilm removal, help eradicate G. vaginalis, reduce the invasion of other potential bacterial pathogens, and therefore lessen the recurrence of bacterial vaginosis.

In one study, patients with a cumulative total of 60 recurrent bacterial vaginosis participated and completed boric acid therapy in addition to nitroimidazole. At week 7 and 12, the cure rates of the combination therapy were 88% and 92% respectively. At week 16, and 28, the cumulative cure rates were 78% and 65% respectively. No adverse reactions were observed for boric acid.

Due to the decline of estrogen levels, middle-aged women tend to show the symptoms of atrophic vaginitis. It is estimated that 10-40 percent of postmenopausal women have symptoms of atrophic vaginitis. Throughout their life cycle, women experience changes in vaginal epithelium influenced by circulating estrogen levels. In childhood, the vaginal epithelium is shallow. Upon puberty, the vaginal epithelium is thickened under estrogen stimulation. Estrogen also promotes the production of abundant glycogen, the substrate Lactobacillus uses to produce lactic acid, which lowers vaginal pH to 3.5˜4.5, forming a natural defensive barrier to prevent vaginal and urinary tract infections. The rise of vaginal pH makes vagina susceptible to Streptococcus, Staphylococcus, E coli, and other bacterial infections.

After menopause, estrogen (primarily estradiol) levels decrease from greater than 120 micrograms per milliliter to about 18 picograms per milliliter. Then there are plenty of cytological changes with the decline of hormone levels, including connective tissue proliferation, elastin disintegration, and glass-like changes of collagen. These changes may lead to granulation, fissures, bruising, telangiectasia and ulcers. Changes happen to not only postmenopausal genital tract, but also urethra. Both vaginal and urinary tract epithelial cells undergo unfavorable changes in postmenopausal women because of their estrogen dependence.

Menopause is the leading cause of decrease in circulating estrogen levels. Therefore, it is the main reason of atrophic vaginitis. In premenopausal women, radiation therapy, chemotherapy, immune disorders and oophorectomy, etc., can suppress ovarian estrogen production. Estrogen levels will also fall in breastfeeding women due to the antagonism of prolactin. The side effects of anti-estrogen drugs, including methylhydroxyprogesterone, tamoxifen, danazol, leuprolide and nafarelin, may also trigger atrophic vaginitis. The severity of atrophic vaginitis is related to the frequency of sexual intercourse, smoking or not, with or without vaginal delivery, whether the patients had vaginal surgery or not, estrogen levels prior to menopause and so on.

The premise of atrophic vaginitis is usually a long-term reduction in estrogen stimulation. A reduction in vaginal lubrication is an early marker of reduced estrogen. Genital Symptoms include skin dryness, burning sensation, pressure feeling, pain during intercourse, lack of vaginal secretions, increased vaginal discharge, and itching. Urinary tract symptoms include urinary discomfort, urinary urgency, hematuria, urinary tract infection, urination difficulties and stress incontinence. Atrophic vaginitis can be combined with Candida infection, trichomonas infection, or bacterial vaginosis. These infections can exacerbate the symptoms of vaginal atrophy. Over time, insufficient vaginal lubrication often leads to sexual dysfunction and emotional distress.

Because the lack of natural estrogen is the primary reason for the emergence of atrophic vaginitis, hormone replacement therapy is the most logical choice. Estrogen replacement therapy can restore normal pH, promote vaginal epithelial thickening and allow the regeneration of vaginal epithelial vessels. Suitable estrogen replacement therapy helps increase the number of superficial cells, alleviate the existing symptoms, and prevent the development of symptoms of the genitourinary system. But contraindications of estrogen therapy include estrogen-sensitive tumors, stage liver failure, and estrogen-dependent thrombosis. Adverse reactions of estrogen therapy are also very prominent, including breast tenderness, increased risk of vaginal bleeding, increased risk of estrogen-dependent tumors, and endometrial cancer and hyperplasia. Additionally, estrogen therapy typically has a long onset time. To completely eliminate dryness, 24 months of therapy may be necessary. Even so, some patients (approximately 10-25%) do not respond to this treatment.

Therefore, reluctance to use estrogen, estrogen therapy contraindications, or adverse reactions during estrogen therapy necessitate a number of patients with atrophic vaginitis to pursue other alternative therapies. Lubricants and moisturizers can help keep vaginal secretion, increase comfort and so on. KY or Astronglide as lubricants can effectively alleviate short-term atrophic vaginitis symptoms, but the action can only last for a few minutes. These lubricants have other drawbacks as they tend to overflow due to poor vaginal retention. The users often feel cold and discomfort upon the first application of these products.

A water-soluble polymer is often used as a carrier for drug delivery system. Bioadhesive drug delivery system utilizes the bioadhesive properties of water-soluble polymers to achieve targeted release of drugs at specific biological sites. Bioadhesion mainly relies on the nature of the bioadhesive agent. The first stage of bioadhesion starts with the intimate contact between the bioadhesive polymer and a biological surface; the second stage of bioadhesion involves the penetration of bioadhesive polymer into epithelial or mucosal tissue. Mucosa network at physiological pH carries negative charges, the high density of charges on the mucous membrane bearing sialic acid and sulfuric acid can significantly reinforce bioadhesion.

Bioadhesive polymers may be used in drug systems for oral, ophthalmic, nasal, dermal, vaginal, and pulmonary delivery, etc. Since these polymer materials have high viscosity, they are also widely used in controlled-release and sustained-release formulations. The sustained release formulations comprising of polycarbophil and chlorothiazide, for example, can be orally administered to rats to continuously release chlorothiazide for up to 8 hours. Polycarbophil offers a gastric retention function, which is attributable to its high viscosity. In one study, duodenal intubation technique was used to determine canine gastric emptying rate. The researchers found that the higher the concentration of polycarbophil, the longer the gastric emptying time. The conclusion was that polycarbophil increased gastric retention via its apparent viscosity. Since polycarbophil contains many carboxylic acid groups, it also has a high degree of pH buffering capacity. In addition, this polymeric material has water swellability. Therefore, it has an extraordinary ability to absorb and retain moisture. As a result, it can be used as a water-soluble lubricant.

CN101773516B disclosed a vaginal gel using polycarbophil and carbomer as acidic agents, methylparaben, ethylparaben and propylparaben sodium salts as alkaline agents, and water and glycerin as solvents. The composition has a pH buffering capacity, playing a role of maintaining vaginal pH.

CN102266283B disclosed a sterile vaginal gel containing polycarbophil and carbomer and V80 or quaternary ammonium compounds for the treatment of vaginitis. CN102688182A disclosed a vaginal pH buffered antimicrobial gel, and its main components are polycarbophil, carbomer, disodium edetate, chitosan, glycerin, triethanolamine, methyl p-hydroxybenzate and deionized water. The disclosure aimed to use the weak acid and chitosan antimicrobial properties to treat vaginitis. In spite of the use of polycarbophil and carbomer as common acidic buffering systems in the above patents, other antimicrobial or antibacterial ingredients varied. The above patents proposed the corresponding solutions for lowering vaginal pH and treatment of vaginitis, but none of them proposed a solution for the resistant vaginitis.

CN101951868B disclosed the use of boric acid as single active pharmaceutical ingredient for the treatment and/or prevention of vaginal infections and/or pathogenic vaginal biofilm. Although the in vitro experiments carried out in this patent proved the efficacy of boric acid on obliterating bacterial biofilm, it did not come up with a boric acid dosage form or formulation for clinical application, nor did it conceive a solution of how to use a practical boric acid formulation to treat ill patients with fungal or bacterial vaginitis.

### BRIEF SUMMARY

The vaginal bioadhesive boric acid formulation disclosed herein are suitable for treating fungal, bacterial, and trichomonal vaginitis, especially for the treatment of recurrent and resistant vaginitis. It may also has a role in regulating vaginal pH value, moisturizing and lubricating. As a result, it may be a treatment option for atrophic vaginitis, complicated or uncomplicated by bacterial or fungal or trichomoniasis infections. The formulation is intended to replace the extemporaneous preparation of boric acid capsules and to provide doctors and patients more manageable, more efficient, more comfortable, more convenient solution to recurrent vaginitis.

Therefore, one embodiment of the present disclosure provides a bioadhesive boric acid formulation that maintains a relative high boric acid concentration (e.g., about 10-20 mg/mL) in vaginal fluid. Because boric acid at a concentration of 10-20 mg/mL inhibited almost all common bacterial or fungal growth, the vaginal bioadhesive boric acid formulation ought to maintain boric acid concentration at similar levels. The boric acid release characteristics can be simulated with in vitro experiments.

Another embodiment of this disclosure provides a method of inhibiting vaginal bacterial and fungal growth, the method comprising vaginally administering a therapeutically effective amount of the bioadhesive boric acid formulation, as described herein. This inhibition efficacy can be proven by in vitro methods.

Another embodiment of this disclosure provides a bioadhesive boric acid formulation that can be used for fungal, bacterial, and Trichomonas vaginitis, especially for treatment of recurrent and resistant vaginitis.

Another embodiment of this disclosure provides a method of treating or alleviating symptoms of atrophic vaginitis (complicated or not complicated with bacterial or fungal or trichomoniasis infections) by vaginally administering a therapeutically effective amount of the bioadhesive boric acid formulation. Advantageously, the vaginal bioadhesive boric acid formulation is capable of adjusting vaginal pH, lubricating and moisturizing vaginal tract.

In various embodiments, the vaginal bioadhesive boric acid formulation may be in the forms of semi-solid preparations, such as gels, cream, ointments, pastes, soft capsules, or other semi-solid dosage forms. The formulation may be delivered directly or through an applicator into the vaginal track.

The vaginal bioadhesive boric acid formulation disclosed herein has a uniform consistency such that there will be no grainy sensation and discomfort for the users; and is

safe to use, without irritation, sensitization, and cytotoxicity.

Another objective of this disclosure is to provide a method for treating patients in need thereof with vaginal bioadhesive boric acid formulation that contains pharmaceutically acceptable boric acid, hydrate and other boric acid derivatives. Methods described herein are meant to give patients the vaginal bioadhesive boric acid formulations within the scope of effective dosage regimen. The general disease conditions include, but not limited to fungal, bacterial, trichomonal vaginitis, atrophic vaginitis, cervicitis, and other gynecological inflammations.

### BRIEF DESCRIPTION OF THE SEVERAL VIEWS OF THE DRAWINGS

FIG. 1 shows a bioadhesive boric acid gel prepared on a laboratory scale.

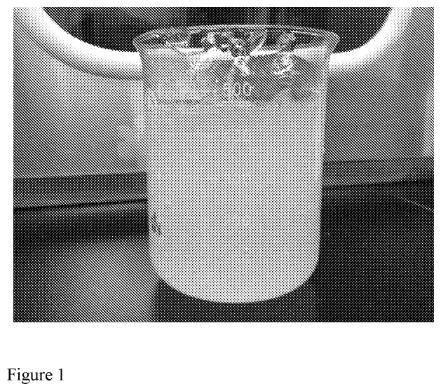

FIG. 2 shows a bioadhesive boric acid gel packaged in a vaginal applicator.

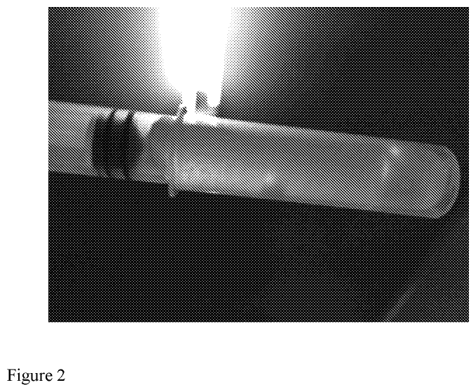

FIG. 3 shows the result of a retention and release study of a bioadhesive boric acid gel according to one embodiment.

FIG. 4 is a calibration curve for boric acid quantification.

FIG. 5 shows the boric acid release profile of a bioadhesive boric acid gel in artificial vaginal fluid.

FIG. 6 shows the antibacterial potency of a bioadhesive boric acid gel.

### DETAILED DESCRIPTION

### Definition and Nomenclature

“Bioadhesive” means two materials (of which at least one is biological) can be held together under interfacial force after a long period of time. “Bioadhesive” means that the carrier systems carrying the drug can be interlocked with a specific biological surface, which may be the upper surface of epithelial tissue or mucosal tissues.

“Bioadhesive polymers” described herein are polymers materials that can serve as binders. They may be derived from a biological monomer, such as a synthetic form of sugar glue, or other synthetic glue designed to adhere to biological tissues.

Bioadhesive polymer materials described herein include, but are not limited to, acacia gum, alginate, carbomer/carbopol, polycarbophil, hydroxypropyl methylcellulose, polycarbophil, hyaluronic acid, pectin, sodium hyaluronate, polyvinyl alcohol, polyvinyl pyrrolidone, and tragacanth.

“Vaginal bioadhesive boric acid formulation” described herein refers to the formulation recipe that can be used to retain active ingredients such as boric acid in the vagina and slowly release the active ingredients over time. This said vaginal formulation plays an important role for vaginal drug delivery because vaginal fluid secretion often causes rapid loss of active ingredient. Such a formulation of the active ingredient can achieve and maintain a certain concentration (e.g., therapeutic level) of the active ingredient and control the release of the active ingredient as to achieve the desired therapeutic effect.

### New Formulation Recipes

In one aspect, the present disclosure provides a vaginal boric acid formulation comprising of 0.1 to 10% (w/v) of boric acid and one or more bioadhesive polymers. The formulation retains and releases boric acid as an antibacterial ingredient in the vaginal fluid. Unless specified otherwise, the concentrations discussed herein refer to weight percentage of the total volume of the formulation (i.e., w/v).

In various embodiments, e.g., Examples 11-16, the present disclosure provides bioadhesive vaginal preparations that contain both boric acid and bioadhesive polymers, including polycarbophil, Carbopol® 934, Carbopol® 940, Carbopol® 974, polyvinyl pyrrolidone, and tragacanth gum.

In a specific, preferred embodiment, the carbomer Carbopol® 974 or polycarbophil act as retention and release matrix for the said vaginal formulations

In a preferred embodiment, the vaginal formulations further contain a biopolymer, e.g., chitosan, for enhanced bacteriostatic effect. Chitosan is a natural antibacterial agent and the concentration of higher than 0.01% of chitosan will produce grainy and course sensation, and affect the uniformity of the formulation. Examples 17-18 further describe in more detail bioadhesife vaginal preparations that contain boric acid, polyacrylic acid polymers (Carbopol 974 and/or polycarbophil), and chitosan.

In various embodiments, e.g., Examples 1-3, the present disclosure provides vaginal preparations that combine boric acid and polyacrylic acid polymers (Carbopol 974 and/or polycarbophil). In particular, the boric acid may be present in an amount of 0.5-5%; and the polyacrylic polymer may be present in an amount of 0.5-3%. In a preferred embodiment, 5% of boric acid is used as an active ingredient of such formulations. Such preparations contain boric acid concentration as high as possible, to enhance the antimicrobial effect of the formulation. However, more than 5% of boric acid is difficult to dissolve in water, resulting in a grainy or coarse texture, which can cause an unpleasant sensation for the user. For preparations of higher concentration of boric acid (e.g., 10%), co-solvents that can facilitate the dissolution of boric acid may be used.

In another preferred embodiment, 1.5% polyacrylic polymer material is used as a retention and release matrix in the formulation. Generally speaking, less than 0.5% of the polyacrylic polymer material formed relatively thin gels because the viscosity is too low. More than 3% of polyacrylic acid polymer material forms a relatively thick gel and may be difficult to apply because the viscosity is too high. Preferably, the formulation has a dynamic viscosity of >100,000 cps.

In a preferred embodiment, the vaginal formulation may further contain glycerol, for enhanced moisturization and lubrication.

In a preferred embodiment, the vaginal formulation may further contain methylparaben, propylparaben, and/or potassium sorbate as a preservative.

In various embodiments, e.g., Examples 19-25, the formulation may be in a semi-solid form and solid vaginal preparations, including creams, ointments, and soft gelatin capsules. These preparations contain a combination of boric acid and polyacrylic acid polymer materials (Carbopol 974 and/or polycarbophil).

In a further embodiment, e.g., example 9, the present disclosure provides a process of preparing a vaginal gel containing boric acid and polyacrylic acid polymer materials (Carbopol 974 and polycarbophil). Such boric formulation can be prepared in a uniform texture and on a large scale suited for clinical research and commercialization.

In a preferred embodiment, this production method comprises first dissolving boric acid in water to provide an aqueous boric acid solution.

In the preferred embodiment, this production method further comprises adding a polymer material with bioadhesive properties to the aqueous boric acid solution portion wise with vigorous stirring.

The suitability of the vaginal bioadhesive boric acid preparations can be evaluated according to the methods described herein. In particular, the ability of the vaginal boric acid formulation to retain and release boric acid as antibacterial ingredient in vaginal fluid can be evaluated in simulated vaginal fluid as described in Example 5-7.

The evaluation may be conducted in an artificial vagina, which may be constructed by using a segment of pig vagina and a condenser with the temperature maintained at 37° C., and a method to utilize vaginal gel retention device containing artificial vagina to determine boric acid release profile.

As exemplified in Example 4, the present disclosure provides the use of methyl cellulose instead of polyacrylic acid polymer material (Carbopol 974 and polycarbophil) to prepare a boric acid gel. The methyl cellulose-based gel is used as a control for demonstrating boric acid retention and release capabilities of polyacrylic polymer material (Carbopol 974 and polycarbophil).

In a preferred embodiment, comparing formulation I, II, III, and IV, using Carbopol 974 and polycarbophil simultaneously in preparing vaginal gel, results in a release of greater than 10 mg/ml of boric acid within 0-2 hours and greater than 2 mg/ml of boric acid thereafter in vaginal fluid. Carbomer 974 and/or polycarbophil used as vaginal gel matrix for boric acid formulation was superior to methyl cellulose, indicating that under the influence of bioadhesive polymer materials, boric acid can stay in the vagina for a prolonged period of time and the release of boric acid during the detention may enable the formulation to achieve the target antibacterial concentration.

In a preferred embodiment, the concentrations of Carbopol 974 and polycarbophil are 0.5% and 1% respectively.

### Function and Usage

In one embodiment, the present disclosure provides a vaginal bioadhesive formulation to maintain a high concentration of boric acid in the vaginal fluid over an extended period, for inhibiting bacteria and mold growth.

In one embodiment, also exemplified in example 8, the present disclosure provides in vitro experiments to evaluate the antibacterial activity of the vaginal bioadhesive boric acid preparation, Formulation I. Vaginal gel significantly inhibited and killed Pseudomonas aeruginosa, Staphylococcus aureus, Escherichia coli, and Candida albicans, demonstrating that vaginal preparations containing boric acid and polyacrylic acid polymer materials can effectively suppress bacteria and mold growth.

The said vaginal bioadhesive boric acid preparations can be used for the treatment of vaginal and cervical diseases, generally including but are not limited to fungal vaginitis, bacterial vaginosis, trichomoniasis, atrophic vaginitis, cervicitis, and other gynecological inflammation. It can also be used for relapsing fungal, bacterial, and trichomonas vaginitis that are resistant to other available treatments.

In another aspect, the present disclosure provides a vaginal bioadhesive boric acid formulation, by regulating vaginal pH, moisturizing and lubricating vagina mucosa, as well as preventing and treating bacterial or fungal or trichomonas infection, which can be useful for preventing or alleviating the symptoms of atrophic vaginitis, with or without the complication of infections.

As described in Example 10, the present disclosure provides the evaluations of appearance, sensory, physical, and chemical properties of vaginal adhesive boric acid formulation of Formulation I. The vaginal adhesive boric acid formulation has a pH between 2-5, similar to the natural physiological pH of the vagina. In a preferred embodiment, the boric acid formulation has a pH value of 3.4. The formulation may thus be used for adjusting the vaginal pH and restoring the natural ability of vagina to resist external invasion of bacteria. Formulation I has a uniform consistency, which affords a smooth sensation to the user, and may play the role of lubricating atrophic vaginitis and ease clinical symptoms of atrophic vaginitis. Formulation I has a uniform and smooth texture, which can avoid causing any discomfort.

In another aspect, the present disclosure provides a vaginal bioadhesive boric acid formulation with a good safety profile, causing no irritation, sensitization, and cytotoxicity.

In embodiment example 10, the present disclosure provides irritation, sensitization, and cytotoxicity tests on Formulation I and the experiments showed that Formulation I was not irritating, sensitizing, and cytotoxic.

In another aspect, the present disclosure provides a bioadhesive vaginal boric acid formulation, containing a pharmaceutically acceptable boric acid, its hydrates and other derivatives, for the treatment of patients with such formulation. The method administers vaginal boric acid formulation described above in the range of effective dosing regimen to treat gynecological diseases. The disease conditions in general include, but are not limited to, fungal vaginitis, bacterial vaginosis, trichomoniasis, atrophic vaginitis, cervicitis, and other gynecological inflammations.

Other objects, advantages, and novel features of the present disclosure are set forth in the above description. Those skilled in the art after reading this patent can understand the patent or learn by practice the true meaning and implications of this disclosure. For these aspects and other aspects not covered, if those skilled in the art after reading this patent could easily learn and apply the idea, concepts, techniques, know-hows, or knowledge taught by this patent without authorization, for product development, production, and marketing, such actions constitute infringement of the present disclosure.

### EXAMPLES

The following embodiment examples are used to further illustrate the disclosure. Embodiment examples are to provide the explanation for the disclosure, rather than to limit the disclosure. Therefore, within the scope of the claims of the disclosure, the simple alterations of the embodiment examples constitute patent infringement.

### Example 1

### Preparation of Formulation I

83 ml purification water was measured and heated to 55° C. To which 5 g boric acid, 0.2 g methylparaben and 0.03 g propylparaben were added while stirring the solution at a speed of 200-300 revolutions/minute for approximately one hour. Once the dissolution of solids was completed, the solution was cooled to room temperature. To this cooled solution, 0.01 g chitosan was added and the mixture stirred for 15 minutes. Thereafter, 0.1 g potassium sorbate and 15 g glycerol were added, and the solids were dissolved by stirring for 5 minutes. Thereafter, 0.5 g carbopol 974 and 1 g polycarbophil were added and stirred for approximately 4 hours until the polymers are fully swollen.

### Example 2

### Preparation of Formulation II

83 ml purification water was measured and heated to 55° C. 5 g boric acid, 0.2 g methylparaben and 0.03 g propylparaben were added while stirring the solution at a speed of 200-300 revolutions/minute for approximately one hour till the solids were fully dissolved before the mixture was cooled to room temperature. 0.01 g chitosan was added and stirred for 15 minutes. 0.1 g potassium sorbate and 15 g glycerol were added and dissolved the solids by stirring for 5 minutes. 1.5 g polycarbophil was added and stirred for approximately 4 hours until the polymers were fully swollen.

### Example 3

### Preparation of Formulation III

83 ml purification water was measured and heated to 55° C. 5 g boric acid, 0.2 g methylparaben and 0.03 g propylparaben was added while stirring the solution at a speed of 200-300 revolutions/minute for approximately one hour till the solids were fully dissolved before the mixture was cooled to room temperature. 0.01 g chitosan was added and stirred for 15 minutes. 0.1 g potassium sorbate and 15 g glycerol were added and the solids was dissolved by stirring for 5 minutes. 1.5 g carbopol 974 was added and stirred for approximately 4 hours until the polymers were fully swollen.

### Example 4

### Preparation of Formulation IV as Control

83 ml purification water was measured and heated to 55° C. 5 g boric acid, 0.2 g methylparaben and 0.03 g propylparaben were added while stirring the solution at a speed of 200-300 revolutions/minute for approximately one hour till the solids were fully dissolved before the mixture was cooled to room temperature. 0.01 g chitosan was added and stirred for 15 minutes. 0.1 g potassium sorbate and 15 g glycerol were added and dissolved the solids by stirring for 5 minutes. 1.5 g methylcellulose was added and stirred for approximately 4 hours until a full swelling of the polymers.

### Example 5

Simulated vaginal fluid was prepared according to the following formula. The pH value was adjusted to 4.2.

| Composition | Simulated vaginal fluid (g/L) |
|---|---|
| Sodium chloride | 3.51 |
| Potassium hydroxide | 1.40 |
| Calcium hydroxide | 0.222 |
| Bovine serum albumin | 0.018 |
| Lactic acid | 2.00 |
| Acetic acid | 1.00 |
| Glycerol | 0.16 |
| Urea | 0.40 |
| Glucose | 5.00 |
| pH value | 4.2 |

### Example 6

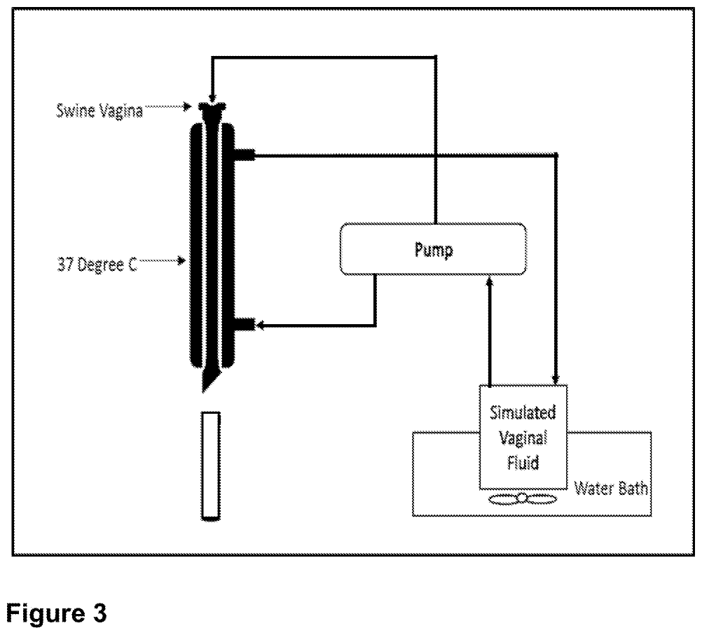

Pig's vagina (procured from a farm and kept at 4° C.) was mounted to a condenser that matched the dimensions of the vagina shown in FIG. 3 (for example, length of the condenser is 11 cm, outer diameter 5 cm, inner diameter 1 to 2.4 cm). 3 g vaginal gel was evenly coated on the inner wall of the vagina. Thereafter, the simulated vaginal fluid was placed in a beaker and preheated in a 37° C. water bath. The preheated simulated vaginal fluid was transferred via a low speed pump at a rate of 3 ml/hour into the condenser attached to the pig's vagina. Meanwhile, vaginal fluid was pumped into the sleeve jacket of the condenser to maintain a temperature of 37° C., simulating human vaginal condition.

The simulated vaginal fluid off the condenser attached with the pig's vagina vaginal fluid was collected with a small tube per sampling time points at 0-10 minutes, 10-20 minutes, 20-30 minutes 50-60 minutes 110-120 minutes 230-240 minutes 470-480 minutes, and 950-960-minute. Approximately 0.5 ml fluid per tube was collected. The boric acid content after dilution of the collected vaginal fluid was then determined.

The results from vaginal 5% boric acid gel with carbomer and polycarbophil (Formulation I), with polycarbophil only (Formulation II), with carbomer only (Formulation III), and methyl cellulose only (control group) were compared in accordance with the above boric acid retention and release testing methods.

### Example 7

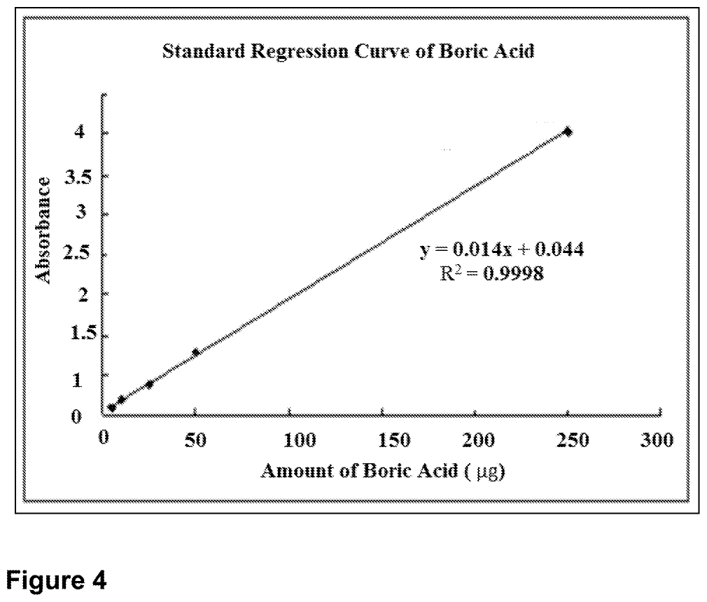

502.4 mg (purity 99.5%) boric acid was accurately weighed into 100 ml volumetric flask and diluted to volume. The solution was diluted in series to achieve target concentrations for linearity standards. 1 mL of linearity standards equivalent to the amounts of 5 μg, 10 μg, 25 μg, 50 μg, and 250 μg boric acid respectively were used for constructing a standard curve. The boric acid amounts were further derivatized per the following method and the UV absorbance using UV-visible spectrophotometer was measured. Calibration curve per UV absorbance was plotted. Results are shown in FIG. 4.

The vaginal fluid collected off the condenser attached with pig's vagina was diluted to 250 ml. 1 ml of the diluted solution was pipetted out for the colorimetric determination of boric acid content with curcumin derivatization.

In particular, for the standard and sample, the following steps were carried out: quantitative pipetted a certain volume (0 to 5 ml) of solution into 25 mL plastic tube and diluted with water to 5 ml. Added sulphuric acid (1+1) solution 1 ml, vortexed and mixed well. Then added 5 ml of ethyl hexanediol/chloroform (1:9) solution, sealed with the lid, and shook for about 2 minutes. Waited until the separation of two layers. Transferred the lower ethyl hexanediol/chloroform solution and filtered through 7 cm diameter filter paper. Transferred 1 ml of the filtrate into 50 mL plastic tubes. Added 1 ml of 0.1% curcumin/glacial acetic acid (w/v) solution and then 0.5 ml of concentrated sulphuric acid, shook and let stand for 30 minutes. Added 25 ml of anhydrous ethanol and let stand for 10 minutes. Performed colorimetric determination of the absorbance at 550 nm with 1 cm curvet.

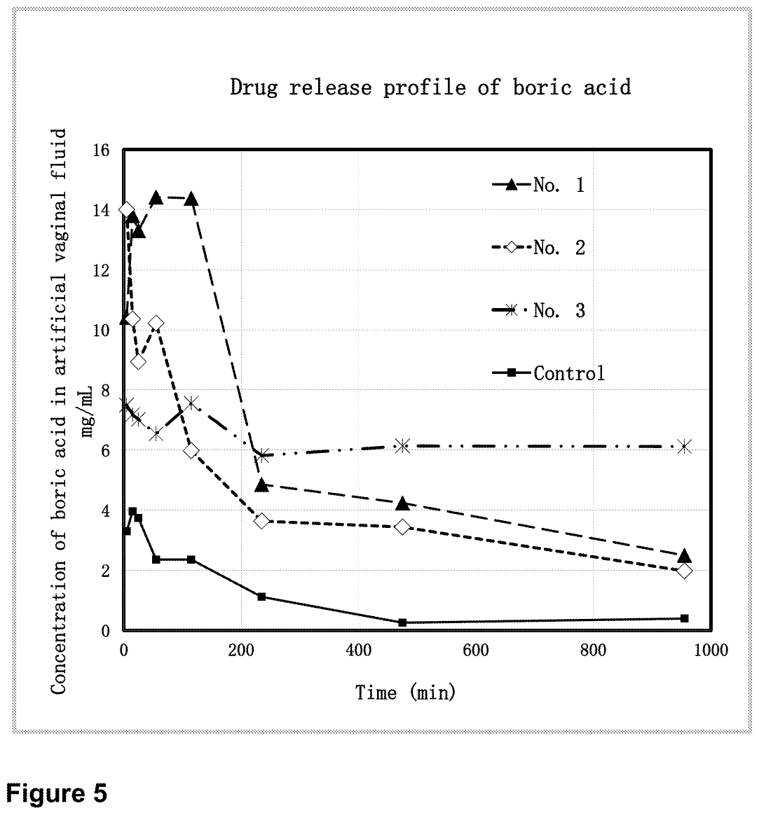

Results are shown in FIG. 5 and the tables below.

Formulation I

| | Tube number | | | | | | | |
|---|---|---|---|---|---|---|---|---|
| | 1 | 2 | 3 | 4 | 5 | 6 | 7 | 8 |
| Absorbance | 0.3357 | 0.4306 | 0.4165 | 0.4475 | 0.4464 | 0.1802 | 0.1630 | 0.1140 |
| Boric acid (mg) | 5.21 | 6.90 | 6.65 | 7.21 | 7.19 | 2.43 | 2.12 | 1.25 |

Formulation II

| | Tube number | | | | | | | |
|---|---|---|---|---|---|---|---|---|
| | 1 | 2 | 3 | 4 | 5 | 6 | 7 | 8 |
| Absorbance | 0.4361 | 0.3341 | 0.2942 | 0.3301 | 0.2115 | 0.1457 | 0.1405 | 0.0993 |
| Boric acid (mg) | 7.00 | 5.18 | 4.47 | 5.11 | 2.99 | 1.82 | 1.72 | 0.99 |

Formulation III

| | Tube number | | | | | | | |
|---|---|---|---|---|---|---|---|---|
| | 1 | 2 | 3 | 4 | 5 | 6 | 7 | 8 |
| Absorbance | 0.2538 | 0.2450 | 0.2408 | 0.2270 | 0.2555 | 0.2067 | 0.2158 | 0.2154 |
| Boric acid (mg) | 3.75 | 3.59 | 3.51 | 3.27 | 3.78 | 2.91 | 3.07 | 3.06 |

Formulation IV

| | Tube number | | | | | | | |
|---|---|---|---|---|---|---|---|---|
| | 1 | 2 | 3 | 4 | 5 | 6 | 7 | 8 |
| Absorbance | 0.1362 | 0.1547 | 0.1486 | 0.1100 | 0.1099 | 0.0755 | 0.0515 | 0.0550 |
| Boric acid (mg) | 1.65 | 1.98 | 1.87 | 1.18 | 1.18 | 0.56 | 0.13 | 0.20 |

### Example 8

The method to culture fresh yeast is shown in the table below. Pseudomonas aeruginosa, Staphylococcus aureus, Escherichia coli, and Candida albicans are liquid cultures. The bacteria were collected by centrifugation, washed with sterile solution of 0.9% sodium chloride and made a suspension of bacteria of about 108 cfu. Agar plate method was used to determine the number of bacteria contained in 1 ml bacterial suspension.

The liquid preparation of bacteria was placed at room temperature and should be used within 2 hours or used within 24 hours if stored at 2-8° C.

| | Test | Culture | | Culture |
|---|---|---|---|---|
| Test strains | medium | Temperature | | Time |
| Staphylococcus aureus | Tryptic | 30~35° | C. | 18~24 hours |
| (CMCC(B)26 003) | soy broth | | | |
| Pseudomonas aeruginosa | Tryptic | 30~3° | C. | 18~24 hours |
| (CMCC(B)10 104) | soy broth | | | |
| Escherichia coli | Tryptic | 30~3° | C. | 18~24 hours |
| (CMCC(B)44102) | soy broth | | | |
| Candida albicans | Saouraud | 20~2° | C. | 24~48 hours |
| (CMCC(F) 98 001) | Dextrose | | | |
| | Broth | | | |

The four types of bacteria as shown in above table were directly inoculated into separate intact packages for testing. For each sample (about 3 grams), the inoculation volume was 20 μl with an inoculation density of 105˜106 cfu/grams. During inoculation, the bacteria were evenly distributed by mixing thoroughly. The samples were stored at 20˜25° C. and kept away from light. Each time, approximately 1 g was taken from the package for testing.

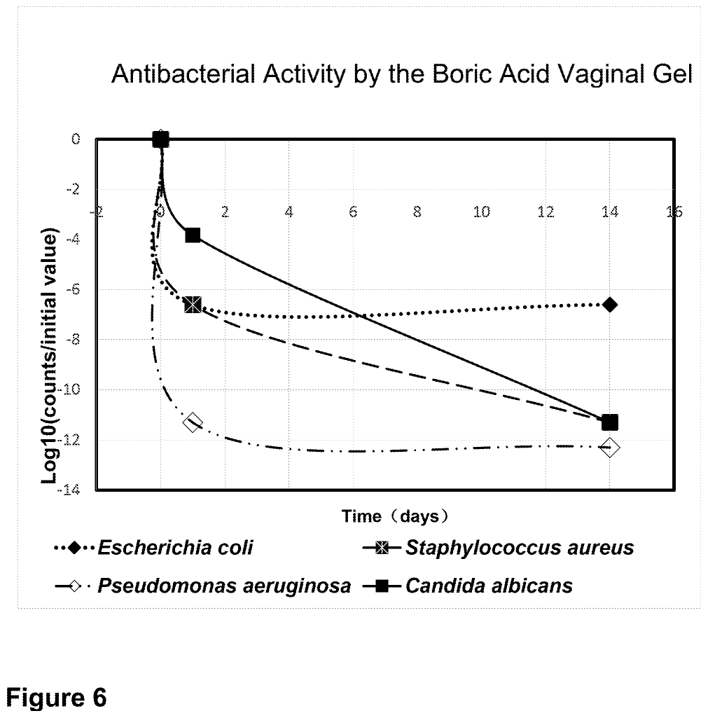

At 0, 14, and 28 days, the content taken out of from the package was diluted to 1000 times and the agar plate method was used to determine bacterial number. The results were shown in the following table and FIG. 6.

| | | Bacteria count (×1000) | | | |
|---|---|---|---|---|---|
| | | Escherichia | Staphylococcus | Pseudomonas | Candida |
| Test Article | Repeat | coli | aureus | aeruginosa | albicans |
| Bioadhesive | 1 | 1 | 1 | 0 | 285 |
| Boric Acid Gel | 2 | 0 | 0 | 0 | 310 |
| Day 0 | | | | | |
| Bioadhesive | 1 | 0 | 0 | 0 | 0 |
| Boric Acid Gel | 2 | 1 | 0 | 0 | 0 |
| Day 14 | | | | | |
| Bioadhesive | 1 | 0 | 0 | 0 | 0 |
| Boric Acid Gel | 2 | 0 | 0 | 0 | 0 |
| Day 28 | | | | | |
| Positive | 1 | ~200-2000 | ~200-2000 | ~200-2000 | ~200-2000 |
| Control Day 0 | 2 | ~200-2000 | ~200-2000 | ~200-2000 | ~200-2000 |

### Example 9

This example describes a process to produce 8 L of Formulation I: To an emulsifying mixer charged with 6640 g purified water, 400 g of boric acid, 16 g of methylparaben, and 2.4 g of propylparaben were added. The heating control panel was set to heat the mixture to 55° C. The mixture was stirred at 50 Hz for 60 minutes until the solids fully dissolve.

The fully dissolved mixture was allowed to cool to below 40° C., whereby 0.8 g chitosan was added into the emulsifying mixer. The mixer was closed and the content was stirred at 50 Hz for 30 minutes. Thereafter, 8 g of sorbic acid and 1200 g of glycerin were added. The mixer was closed and the content was stirred at 50 Hz for 5 minutes.

To this mixture, 40 g carbopol 974P NF powder, which had been previously filtered through 20 mesh sieve, was added while stirring at 50 Hz. When the powder was all added into the mixer, pumped down to vacuum until the pressure gauge reading was approximately 0.08 MPa. The content was stirred for 120 minutes until the full swelling of carbopol 974P NF was reached.

About two hours later, 80 g of polycarbophil (NOVEON® AA-1, filtered through 20 mesh sieve) was added. The mixer was closed and the content was stirred at 50 Hz. When the powder was all added into the mixer, pumped down to vacuum until the pressure gauge reading was approximately 0.08 MPa. The content was stirred for 120 minutes until the full swelling of polycarbophil was reached.

After the stirring was complete, the final product was filled into a vaginal applicator or any suitable gel filling apparatus. The gel filling apparatus, including the hopper and pipe, was sanitized and disinfected by cleaning with purified water followed by 75% of medical alcohol. The pressure regulator was adjusted until the pressure gauge reading was 0.4 MPa; and the metering valve was adjusted to the desired filling quantity (e.g., 3 g per applicator). To fill, rotate the operation switch to manual mode; line up the feeding inlet of the vaginal applicator and loading outlet of the filling apparatus; and switch on the filling apparatus to fill the gel product into the vaginal applicator. The filling quantity could be suitably controlled. The applicator was sealed after filling; labeled and packaged into a secondary product box and crate.

### Example 10

The appearance, physical properties (viscosity), chemistry (pH value), and safety (cytotoxicity, delayed hypersensitivity, and vaginal irritation) of the bioadhesive boric acid gel was evaluated. The results are shown as follows:

| Test Items | Acceptance criteria | Results |
|---|---|---|
| Appearance | Transparent or white semisolid, | Conforms |
| | uniform and fine, stable as a gel | |
| | without drying or liquefying at | |
| | room temperature | |
| Dynamic viscosity | Not less than 1000 mPa · s | 130370 |
| pH value | pH value 2-5 | 3.4 |
| Delayed | not more than level 2 | Level 1 |
| hypersensitivity | | |
| Delayed | No delayed hypersensitivity reaction | Conforms |
| hypersensitivity | | |
| Vaginal irritation | No vaginal irritation reaction | Conforms |

### Example 11

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g polycarbophil was added and stirred for approximately 4 hours until the full swelling of polycarbophil was reached.

### Example 12

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g Carbopol 934 was added and stirred for approximately 4 hours until the full swelling of Carbopol 934 was reached.

### Example 13

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g Carbopol 940 was added and stirred for approximately 4 hours until the full swelling of Carbopol 940 was reached.

### Example 14

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g Carbopol 974 was added and stirred for approximately 4 hours until the full swelling of Carbopol 974 was reached.

### Example 15

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g pyrrolidone was added and stirred for approximately 4 hours until the full swelling of pyrrolidone was reached.

### Example 16

83 ml purified water was measure and heated to 55° C. While stirring and mixing (speed of 200-300 revolutions/minute), 5 g boric acid was added. The mixture was stirred for approximately 1 hour before being cooled to room temperature. 15 g glycerol was added and stirred for 5 minutes. 1.5 g tragacanth gum was added and stirred for approximately 4 hours until the full swelling of tragacanth gum was reached.

### Example 17

83 ml purified water was measure and heated to 55° C. While stirring and mixing (speed of 200-300 revolutions/minute), 5 g boric acid was added. The mixture was stirred for approximately 1 hour before being cooled to room temperature. 0.01 g chitosan was added and stirred for 15 minutes. Thereafter, 15 g glycerol was added and stirred for 5 minutes. 1.5 g polycarbophil was added and stirred for approximately 4 hours until the full swelling of polycarbophil was reached.

### Example 18

83 ml purified water was measure and heated to 55° C. While stirring and mixing (speed of 200-300 revolutions/minute), 5 g boric acid was added. The mixture was stirred for approximately 1 hour before being cooled to room temperature. 0.01 g chitosan was added and stirred for 15 minutes, followed by adding 0.1 g potassium sorbate. Thereafter, 15 g glycerol was added and stirred for 5 minutes. 1.5 g polycarbophil was added and stirred for approximately 4 hours until the full swelling of polycarbophil was reached.

### Example 19

83 ml lotion preparation (such as commercially available fragrance-free lotion under the trade name JERGENS®) was measured and heated to 55° C. While stirring and mixing (speed of 200-300 revolutions/minute), 5 g boric acid was added. The mixture obtained was stirred for approximately 1 hour. 0.5 g polycarbophil was then added The mixture was cooled to room temperature and stirred for approximately 1 hour until a uniform lotion was obtained.

### Example 20

83 ml purified water was measure and heated to 55° C. While stirring and mixing (speed of 200-300 revolutions/minute), 5 g boric acid was added. The mixture was stirred for approximately 1 hour before being cooled to room temperature. 0.01 g chitosan was added and stirred for 15 minutes, followed by adding 0.1 g potassium sorbate. Thereafter, 15 g glycerol was added and stirred for 5 minutes. 1.5 g polycarbophil was added and stirred for approximately 4 hours until the full swelling of polycarbophil was reached. The resulting gel was poured into tampons used for the mucous membrane.

### Example 21

The following steps were carried out to prepare a suppository preparation: Weighed approximately 20 g of PEG800, 60 g of PEG4000, heated to 80° C. While stirring and mixing (speed of 200-300 revolutions/minute), added 5 g boric acid. Stirred for approximately 1 hour. Transferred to the suppository mold. Cooled to room temperature, or cooled in the refrigerator until solidified, then ejected the suppository for packaging.

### Example 22

The following steps were carried out to prepare an emulsion preparation: Measured 83 ml purified water. Heated it to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and then 15 g glycerol and stirred for 5 minutes. Added 0.5 g polycarbophil. Meanwhile, added 1.5 g bee wax into 10 ml mineral oil. Added 2 g glycerol monostearate, 0.2 g propylparaben and heated it to 55° C. Mixed aqueous and oil phases for approximately 4 hours until a uniform emulsion is obtained.

### Example 23

The following steps were carried out to prepare a capsule preparation: Weighed approximately 5 g of boric acid, 0.01 g of chitosan, and 1.5 g of polycarbophil. Mixed the above for approximately 1 hour until a uniform powder is obtained. The powder can be used directly for vaginal application, or poured into a capsule for vaginal application.

### Example 24

The following steps were carried out to prepare a tablet preparation: Weighed approximately 5 g of boric acid, 0.01 g of chitosan, 5 g of microcrystalline cellulose, 10 g of lactose, 0.5 g of sodium carboxymethylcellulose, 0.1 g of magnesium stearate, and 1.5 g of polycarbophil. Mixed for approximately 1 hour until a uniform powder is obtained. The powder was then punched into tablet with tablet compression machine for vaginal application.

### Example 25

The following steps were carried out to prepare a semisolid preparation: Measured 30 ml of PEG400, 50 ml of propylene glycol, 10 ml of glycerol medium chain triester, and 1 ml of polysorbate. Heated the mixture to 55° C. While stirring and mixing (speed of 200-300 revolutions/minute), added 5 g boric acid. Stirred for approximately 1 hour. Cooled to room temperature. Added 0.01 g chitosan and stirred for 15 minutes. Added 0.1 g potassium sorbate and 1.5 g polycarbophil. Stirred for approximately 4 hours until the full swelling of the polymer and a uniform semisolid was obtained. Filled the semisolid into soft gel for vaginal application.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of inhibiting vaginal bacterial or fungal growth in a subject in need thereof, the method comprising vaginally administering a therapeutically effective amount of a bioadhesive boric acid formulation comprising 0.1-10% boric acid and one or more bioadhesive polymers.

2. The method according to claim 1, wherein the one or more bioadhesive polymers are polyacrylic acid polymers.

3. The method of claim 2 wherein the polyacrylic acid polymers are carbopol or polycarbophil.

4. The method according to claim 1, wherein the bioadhesive boric acid formulation further comprises chitosan.

5. The method according to claim 1, wherein the biadhesive boric acid formulation further comprise polyvinyl pyrrolidone, polyethylene glycols, gums, or a mixture thereof.

6. The method according to claim 5, wherein polyethylene glycols include PEG 800, PEG 4000, PEG 2000 or a mixture thereof; and wherein gums include tragacanth gum, locust bean gum, Arabic gum, and gellan gum, or a mixture thereof.

7. The method according to claim 1, further comprising one or more moisturizing agents.

8. The method of claim 7 wherein the one or more moisturizing agents include glycerin.

9. The method according to claim 1, wherein the formulation is a semisolid or solid preparation selected from the group consisting of gel, cream, paste, ointment, suppository, soft capsule, and hard capsule.

10. The method according to claim 1 wherein the vaginal bacterial or fungal growth is associated with a gynecological inflammation disease selected from the group consisting of fungal infection, bacterial infection, trichomoniasis infection, atrophic vaginitis, and cervicitis.

11. The method according to claim 1 wherein boric acid is present in the bioadhesive boric acid formulation in a concentration of 5%.

12. The method according to claim 1 wherein the one or more bioadhesive polymers are present in the bioadhesive boric acid formulation in a concentration of 0.5%-3%.

13. The method of claim 1 wherein the bioadhesive boric acid formulation has a dynamic viscosity >100000 cps.

14. The method of claim 1 wherein the bioadhesive boric acid formulation has a pH value in the range of 2-5.

15. A method for treating or alleviating symptoms of atrophic vaginitis, the method comprising vaginally administering a therapeutically effective amount of a bioadhesive boric acid formulation comprising 0.1-10% boric acid and a bioadhesive gel matrix having one or more bioadhesive polymers.

16. The method according to claim 15, wherein the one or more bioadhesive polymers are polyacrylic acid polymers.

17. The method of claim 16 wherein the polyacrylic acid polymers are carbopol or polycarbophil.

18. The method according to claim 15, wherein the bioadhesive boric acid formulation contains further comprises chitosan.

19. The method according to claim 15 wherein boric acid is present in the bioadhesive boric acid formulation in a concentration of 5%, and the one or more bioadhesive polymers are present in the bioadhesive boric acid formulation in a concentration of 0.5%-3%.

20. The method of claim 15 wherein boric acid is slowly released from the bioadhesive gel matrix over an extended release period.
